(19) United States
(12) Patent Application Publication
(10) Pub. No.: US 2016/0243298 A1
(43) Pub. Date: Aug. 25, 2016

(54) Blood treatment apparatus with increased patient safety and method

(71) Applicants: FRESENIUS MEDICAL CARE DEUTSCHLAND GMBH, Bad Homburg (DE); FRESENIUS MEDICAL CARE AG & CO. KGAA, Bad Homburg (DE)

(72) Inventors: Gerhard Weisen, Bad Homburg (DE); Thomas Pohl, Friedrichsdorf (DE); Jenspeter Rau, München (DE)

(21) Appl. No.: 15/033,447

(22) PCT Filed: Oct. 31, 2014
(86) PCT No.: PCT/EP2014/073452

§ 371 Date: Apr. 29, 2016

(30) Foreign Application Priority Data
Oct. 31, 2013 (DE) 10 2013 112 038.5

(51) Int. Cl. A61M 1/16

(52) U.S. Cl. CPC A61M 1/1603; A61M 1/168; A61M 2205/15; A61M 2205/18

(57) ABSTRACT

A blood treatment apparatus with a dialysis filter or with an intake for a dialysis filter, a dialysis liquid inlet line with a first connector to feed dialysis liquid into the dialysis filter, a dialysate outlet line with a second connector to discharge dialysate from the dialysis filter, a blood leak sensor which is configured and arranged to detect blood in or at the dialysis filter, in the dialysis liquid inlet line and/or in the dialysate outlet line, and configured to emit a blood leak signal (BLS) if blood is detected, a short-circuit line to connect the dialysis liquid inlet line and/or with the dialysate outlet line in fluid communication, wherein the short-circuit line comprises for this purpose a third connector and a fourth connector to connect with one of the first and second connector, and a control circuit, respectively.

### CROSS REFERENCE TO RELATED APPLICATIONS

The present application is the national stage entry of International Patent Application No. PCT/EP2014/073452, filed on Oct. 31, 2014, the disclosure of which is expressly incorporated herein by reference in its entirety, and which claims priority to German Application DE 10 2013 112 038.5, filed on Oct. 31, 2013.

### FIELD OF INVENTION

The present invention relates to a blood treatment apparatus, a method of operating a blood treatment apparatus, and a method for replacing a dialysis or blood filter of a blood treatment apparatus.

### BACKGROUND

From practice, blood treatment apparatuses with blood filters or dialysis filters are known. By means of the dialysis filter, both blood and dialysis liquid are lead into compartments, which are separated by means of a dialysis membrane, the blood compartment and the dialysate compartment. The dialysis filter is both connected to the patient by means of an extracorporeal blood circuit and connected to the blood treatment apparatus by means of a feeding dialysis liquid line and a discharging dialysate outlet line which both comprise each one connector to be connected with the dialysate outlet line dialysis filter. This is referred to as dialysate side and blood side.

In very rare cases, blood can transit from the blood compartment to the dialysate compartment after rupture of the dialysis membrane. This is called a blood leak. Such a blood leak causes the transition of blood into the dialysate compartment and thus to the dialysate side. Escaping blood is detected by means of a (normally optical) sensor on the dialysate side. As a consequence an alarm message (optically as message on a display and if applicable also acoustically) is emitted. The medical personnel in charge may suspend such alarm message by user input for a predetermined period, for example 2 minutes, during which the treatment is continued. This is based on the experience that smaller ruptures of the dialysis membrane may close themselves and thus the treatment may be safely continued.

If the blood leak is still present after this suspension time period (to be detected again by means of the corresponding sensor signal at the blood leak detector) the operating personnel can be requested to interrupt the treatment and change or replace the blood filter or dialysis filter.

When replacing the dialysis filter the used dialyzer has to be emptied. Thereby, the dialysis liquid inlet line and the dialysate outlet line are temporarily connected with a short-circuit line or a short-circuit unit of the blood treatment apparatus. The temporarily loose dialysis liquid inlet line and the temporarily also loose dialysate outlet line are so to say parked at one connector of the short-circuit line, respectively. Thus, their connectors on the one hand do not get contaminated by their environment and on the other hand, the personnel have their hands free to change or replace the dialysis filter.

The current way of replacing the dialyzer holds the risk of a contamination, by patient blood, of the connectors of the dialysate inlet line and/or the dialysate outlet line, which are mostly designed as Hansen couplings, and of the connections of the short-circuit unit or the short-circuit line. This is the case if or when from a blood leak blood from the extracorporeal blood circuit has transited to the dialysate side and thus has reached the connectors of the short-circuit line.

Through the intermediate replugging of the connectors of the dialysate inlet line and/or the dialysate outlet line to the connectors of the short-circuit line it can also be contaminated, including its connectors or connecting pieces. This contamination may affect both the interior of the lines and the connectors. It may further affect the inner surface of the connectors and thus, after connection of the connectors of the dialysate inlet line and/or the dialysate outlet line with those of the short-circuit line, also their contact surface.

Such contamination has an impact on the later treatment of a further patient by means of the same blood treatment apparatus. If an extremely thorough cleaning procedure beyond the usual cleaning programs does not take place, the dialysis liquid inlet line and consequently also the dialysate compartment of the dialysis filter may get contaminated with pathogens and other substances from the blood of the patient in treatment at the time of the rupture. If the pathogens or substances in question are membrane-permeable, they may also transit across the membrane into the blood of future patients in treatment. This must be prevented as far as possible.

### SUMMARY

One object of the present invention is to propose a blood treatment apparatus, which reduces the contamination risk in the case of a blood transition to the dialysate side. Additionally, suitable methods to reduce the contamination risk are to be specified.

This object according to the invention may be accomplished by means of the blood treatment apparatus and methods according to the present invention.

Thus, according to the present invention a blood treatment apparatus is proposed which optionally comprises a dialysis filter and/or an intake for a dialysis filter. It further comprises a dialysis liquid inlet line with a first connector, which is preferably located at the end to feed dialysis liquid into the dialysis filter and to be connected with it. It also comprises a dialysate outlet line with a second connector, which is preferably located at the end to discharge dialysate from the dialysis filter and to be connected with it.

The blood treatment apparatus further comprises a blood leak sensor, which is configured and arranged to detect blood in or at the dialysis filter, in the dialysis liquid inlet line and/or in the dialysate outlet line. It is further configured to emit a blood leak signal if blood is detected.

The blood treatment apparatus comprises a short-circuit line to connect the dialysis liquid inlet line and the dialysate outlet line (or with the dialysate outlet line, i.e. directly or by means of an intermediate element) in fluid communication. The short-circuit line comprises a third connector and a fourth connector for this purpose, each configured to connect to one of the first and the second connector, respectively.

Furthermore, the blood treatment apparatus comprises a control circuit which alternatively may be designed as a regulation circuit, and which is configured to emit at least a warning or a hint to the user that a cleaning procedure of the third and/or fourth connector is necessary, to start a prescribed cleaning procedure of the third and/or fourth connector, to stop or prohibit the operability of the blood treatment apparatus until the user has completed one or more prescribed activities, and to mechanically prevent an initiation of connection of the third and/or fourth connector with the first and/or second connector if or when a blood leak signal has been emitted and/or received and/or processed. When “emit” is mentioned hereinafter, it may also be optionally understood in certain embodiments according to the present invention as “receive” or “process”.

The method according to the present invention to operate a blood treatment apparatus encompasses providing a blood treatment apparatus which comprises a dialysis filter or an intake for a dialysis filter, a dialysis liquid inlet line with a first connector to feed dialysis liquid into the dialysis filter as well as a dialysate outlet line with a second connector to discharge dialysate from the dialysis filter. The blood treatment apparatus further comprises a blood leak sensor which serves to detect blood in or at the dialysis filter, in the dialysis liquid inlet line and/or in the dialysate outlet line, and to emit a blood leak signal if blood is detected. Furthermore, it comprises a short-circuit line to connect the dialysis liquid inlet line and—or with—the dialysate outlet line in fluid communication, wherein the short-circuit line comprises a third connector and a fourth connector to connect with one of the first and the second connector, respectively, and a control circuit.

The method encompasses detecting by means of a blood leak sensor whether a blood leak is present, and emitting a blood leak signal, if this is the case. Optionally, the blood leak signal can be buffered in a storage device. The method encompasses further checking whether a blood leak signal has been emitted and/or received and/or processed and initiating a step if a blood leak signal has been emitted and/or received and/or processed, from the group which consists of emitting a warning to the user that cleaning the third and/or fourth connector is required or necessary, starting a prescribed cleaning procedure of the third and/or fourth connector, stopping or prohibiting the operability of the blood treatment apparatus until the user has completed at least one prescribed activity and mechanical prevention of a connection of the third and/or fourth connector with the first and/or second connector.

The method according to the present invention for replacing or changing a dialysis filter of a blood treatment apparatus encompasses providing a blood treatment apparatus which comprises a first dialysis filter in an intake for a dialysis filter, a dialysis liquid inlet line with a first connector to feed dialysis liquid into the first dialysis filter as well as a dialysate outlet line with a second connector to discharge dialysate from the first dialysis filter. It further comprises a blood leak sensor, which serves to detect blood in or at the first dialysis filter, in the dialysis liquid inlet line and/or in the dialysate outlet line, and to emit a blood leak signal if blood is detected. It further comprises a short-circuit line to connect the dialysis liquid inlet line and—or with—the dialysate outlet line in fluid communication, wherein the short-circuit line comprises a third connector and a fourth connector to connect with one of the first and second connector, respectively, for this purpose.

The method encompasses further providing a second dialysis filter, separating the dialysis liquid inlet line and the dialysate outlet line from the first dialysis filter and connecting the dialysis liquid inlet line and the dialysate outlet line with the second dialysis filter, without having previously connected the dialysis liquid inlet line or the dialysate outlet line with the short-circuit line.

In all of the following embodiments, the use of the expression may be or may have and so on, is to be understood synonymously with preferably is or preferably has, respectively, and so on, and is intended to illustrate an embodiment according to the present invention.

Whenever numerical phrases are mentioned in here, the person skilled in the art understands this as an indication of a lower numerical limit. If this does not lead to a contradiction recognized by the skilled person, the skilled person thus always also reads at least one or at least a when the indication one or a, as an example, is given. This type of understanding is also encompassed by the present invention, just as the interpretation that a numerical phrase as for example one may alternatively be meant as exactly one, wherever this is obviously technically feasible in view of the person skilled in the art. Both is encompassed by the present invention and applies to all numerical phrases used herein.

Embodiments according to the present invention may comprise one or more of the features named hereafter, in arbitrary combination.

In certain exemplary embodiments according to the present invention, initiating means prompting or emitting a required signal, which starts or exerts the desired step in interaction with the blood treatment apparatus.

In certain exemplary embodiments according to the present invention, detecting blood components and/or detecting a change in dialysate, which allows a conclusion on a present blood leak, also counts among the detection of blood. The change may for example be a property, which is measurable by means of optical sensors.

In some exemplary embodiments according to the present invention, the blood treatment apparatus comprises a monitoring device which is configured to detect a connecting of the third and/or the fourth connector, for example with the first and/or second connector. It is further configured in these embodiments to emit a connection signal if connecting is detected. The control circuit is thereby configured to initiate at least one step from the above-mentioned group, if, for example within a predetermined period, both a blood leak signal and a connection signal have been emitted and/or received and/or processed.

In some exemplary embodiments according to the present invention, the control circuit is configured to initiate at least one step from the above-mentioned group, if, and only if, both a blood leak signal and a connection signal have been emitted and/or received and/or processed.

In certain exemplary embodiments according to the present invention of the blood treatment apparatus, the monitoring device comprises or consists of a connection sensor. The connection sensor is configured and arranged to detect a successfully taken place or established connection with the third and/or with the fourth connector. It is furthermore configured to emit a connection signal if connecting is detected.

In some exemplary embodiments of the blood treatment apparatus, the monitoring device is or comprises a query device by means of which the user has to confirm or deny a successful connection upon a query, for example by means of a display or a touch screen. Alternatively, the blood treatment apparatus may comprise the query device.

In some exemplary embodiments according to the present invention, the treatment of a patient by means of the blood treatment apparatus cannot be continued or resumed if the user confirms that such successful connection has taken place. Alternatively or additionally, a warning may be emitted to keep the treatment suspended or not to start the treatment of a further patient or the next patient. The blood treatment apparatus and/or the devices, in particular the blood treatment apparatus, may be correspondingly set, programmed or configured. The terms mentioned last shall be understood as synonyms in certain embodiments according to the present invention.

In certain exemplary embodiments according to the present invention, the prescribed action is chosen from a group which consists of confirming, for example by means of a touch display, and executing cleaning steps by the user, preferably with subsequent confirmation by the user that the cleaning procedure has taken place, or the group encompasses arbitrary combinations hereof or the like.

In some exemplary embodiments according to the present invention, the prescribed activity is a pre-determined activity. Its definition, description or the like may, e.g., be as usual deposited or saved in suitable devices, which may be, e.g., part of the blood treatment apparatus or connected thereto.

In certain exemplary embodiments according to the present invention, the prescribed activity is not switching off an alarm, in particular not a sole switching off.

In some exemplary embodiments according to the present invention, the blood treatment apparatus comprises further an interlock or cover which in an interlock or cover state hereof covers at least the third and/or the fourth connector in a cover state such that a connecting of the at least one covered connector with the first or the second connector is not possible. The interlock is—preferably manually—transferable into a connection state hereof in which connecting is possible. Thereby, the mechanical prevention of connecting of the third and/or fourth connector with the first and/or second connector is a transfer into or a retaining in an interlock state or cover state. Thereby, the interlock or cover can be secured against unauthorized opening or transferring into the connection state, for example by determining the forces necessary herefor as sufficiently high enough. One option is to allow opening or transferring only by destruction of components or only by giving a password which has been disclosed only to a limited group of people, by way of example.

In some exemplary embodiments according to the present invention of the blood treatment apparatus, the cover is a flap.

In certain exemplary embodiments according to the present invention, the blood treatment apparatus comprises a storage device which is configured or controllable to buffer the blood leak signal which is emitted and/or received and/or processed only in the case of a blood leak for a predetermined period, for example until the completion of the blood treatment or until it is deleted by the user. The predetermined period may last until the occurrence of a predetermined event, in particular the start of a treatment of the next patient, and may be terminated with the occurrence of the predetermined event.

In some exemplary embodiments according to the present invention, the blood treatment apparatus comprises a monitoring device to detect a successful connection of the third and/or the fourth connector, for example with the first and/or second connector, and to emit a connection signal. Thereby, the method comprises detection by means of the monitoring device whether after the output of the blood leak signal the third and/or the fourth connector has been connected. A connection signal is emitted if this is the case. Thereby, the control circuit is configured to initiate at least one step from the above-mentioned group if, and only if, both a blood leak signal and a connection signal have been emitted and/or received and/or processed.

In some exemplary embodiments according to the present invention, both connectors of the short-circuit line are always covered by a flap (short-circuit unit flap) when they are not connected. A sensor detects any opening of the flap. This is another possible way of monitoring whether the third or fourth connector has possibly been connected. For simplicity reasons, it is assumed here that a connection has at least theoretically been possible in view of the demonstrably at least in the meantime open flap.

In certain exemplary embodiments a possible way of monitoring is checking whether after a blood leak signal a fluid flow could be measured in the short-circuit line. If there has been such flow, it is simply assumed that the third and the fourth connector will have been connected with connectors of the dialysis liquid inlet line and the dialysate outlet line.

Embodiments according to the present invention may comprise one or more advantages named herein. Among these count especially the ones hereafter.

According to the present invention, the safety of a subsequent patient can be increased as a connection of contaminated connectors with the short-circuit line is or will be detected and countermeasures can be taken. In some embodiments according to the present invention, the safety achieved can even be increased as the present invention includes that countermeasures for the prevention or elimination of the contamination indispensably have to be taken as otherwise the blood treatment apparatus is not usable for further patients for the time being.

If the user does not plug the dialysis liquid inlet line and the dialysate outlet line onto the connectors of the short-circuit line as proposed according to the present invention but instead directly from the old, first dialysis filter to the new dialysis filter when a blood leak occurs with subsequent replacement of the dialysis filter or dialyzer, it is reliably prevented that the short-circuit line or other components of the blood treatment apparatus are contaminated.

But even in the case that the user has not adhered to this approach as recommended according to the present invention for replacing the dialysis filter with the blood leak, protection against contamination is provided as the blood treatment apparatus according to the present invention recognizes (for example, by setting a corresponding flag) that after a blood leak alarm the dialysis liquid inlet line and the dialysate outlet line were plugged onto the short-circuit line, for example as discussed above in that corresponding sensors detect a connection or also by a corresponding entry the user makes into the user interface of the dialysis machine (for example, by issuing a corresponding question on the query device such as for example the display: Have the dialysate inlet line and/or the dialysate outlet line been plugged onto the short-circuit unit? Options via touch button yes, no) after the replacement of the dialysis filter.

Depending on the action taken by the user, i.e. depending on whether the user has plugged the dialysis liquid inlet line and/or the dialysate outlet line onto the connectors of the short-circuit line after a blood leak alarm, an appropriate cleaning procedure is requested before the next treatment in order to ensure that all possible contamination spots are disinfected. This may encompass immersing the connectors into disinfectant solution, surface disinfection of the connectors of the short-circuit line and more.

The blood treatment apparatus according to the present invention may record these steps by issuing questions to the user and expected entries without which no treatment is possible with an accordingly configured control circuit. This way, the user personnel is advantageously alerted and sensitized for the special situation (blood leak in the preceding treatment).

The present invention is hereafter described with reference to the purely exemplary, non-restrictive embodiments of the appended figures. In the figures, identical reference numerals refer to the same or identical elements.

### BRIEF DESCRIPTION OF THE DRAWINGS

FIG. 1 shows in a schematically very simplified way sections of a blood treatment apparatus according to the present invention in a first embodiment.

FIG. 2 shows in a schematically very simplified way sections of a blood treatment apparatus according to the present invention in a second embodiment and in a connection state.

FIG. 3 shows the sections of the blood treatment apparatus from FIG. 2 in a cover or interlock state.

FIG. 4 shows the method according to the present invention in an exemplary embodiment.

### DETAILED DESCRIPTION

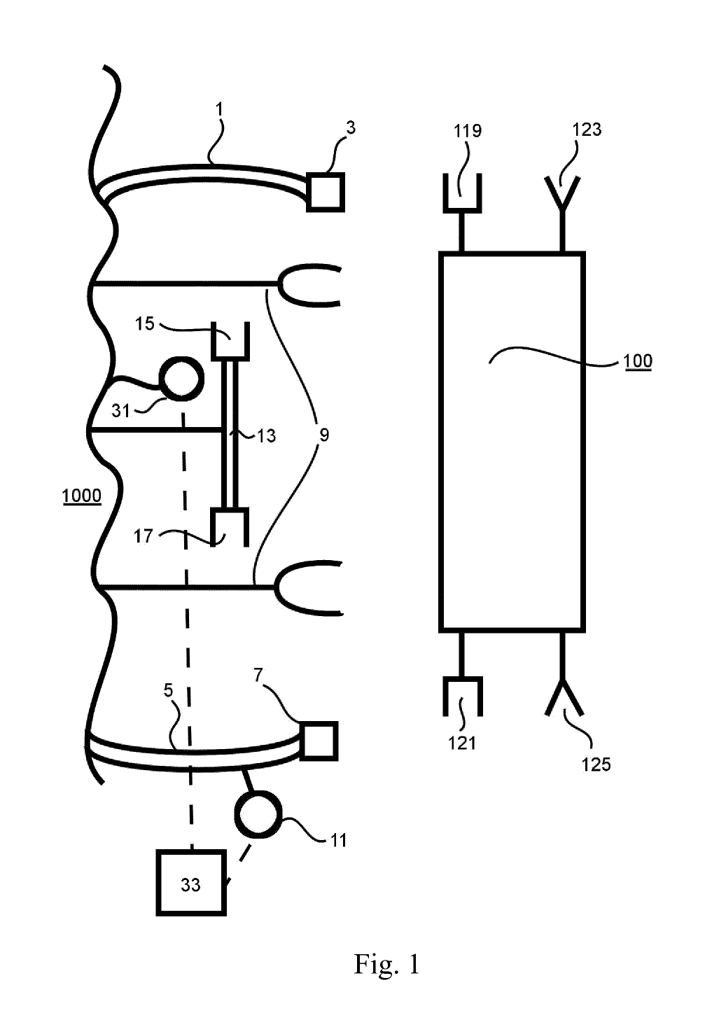

FIG. 1 shows in a schematically very simplified way sections of a blood treatment apparatus 1000 according to the present invention in a first embodiment.

The blood treatment apparatus 1000 comprises a dialysis liquid inlet line 1 with a first connector 3 and a second dialysate outlet line 5 with a second connector 7. Both the first and the second connector 3, 7 may optionally be designed as Hansen couplings or other types of connectors.

The blood treatment apparatus 1000 further comprises an, optionally multi-part, intake 9 to hold a dialysis filter 100 with a blood side and a dialysate side in a detachable way. The dialysate outlet line 5 comprises a blood leak sensor 11 to detect blood in the interior of the dialysate outlet line 5. However, the blood leak sensor 11 may be provided alternatively or additionally at any other location at which the transition of blood from the blood side of the dialysis filter 100 to its dialysate side might be detected.

Furthermore, the blood treatment apparatus 1000 comprises a short-circuit line 13 with a third connector 15 and a fourth connector 17. Both the third and the fourth connector 13, 17 may optionally be designed as part or counterpart to Hansen couplings or as other types of connectors.

The dialysis filter 100 also comprises connectors 119, 121 which are designed to be connected with the first and second connector 3, 7. Furthermore, the dialysis filter 100 comprises connectors 123, 125, which are not relevant here, to be connected with the arterial and the venous line.

For the treatment of the patient the connectors 119, 121 of the dialysate side of the dialysate filter 100 can be connected with the dialysate filter 100 via the first and the second connector 3, 7 as is usual and required during and for the patient treatment.

In certain situations like changing or replacing the dialysis filter 100 the first and the second connector 3, 7 have to be detached from the dialysis filter 100. The dialysis filter 100 is thus uncoupled from the blood treatment apparatus 1000. The first and the second connector 3, 7 are subsequently connected in fluid communication with the third connector 15 and the fourth connector 17 of the short-circuit line 13. In this position, lines 1 and 5 could, if desired, even be flushed if or when the dialysis filter 100 is uncoupled.

However, in the event that by means of the blood leak sensor 11 blood is detected on the dialysate side, here mainly in the dialysate outlet line 5, the first and second connector 3 and 7 are also to be detached from the dialysis filter 100 so it can be changed or replaced. While the latter is changed or replaced, the first and second connector 3 and 7 are not plugged upon the third connector 15 and the fourth connector 17 of the short-circuit line 13 or connected therewith in the case of a blood alarm, and lines 1 and 5 must not be flushed by means of the short-circuit line 13.

To ensure that lines 1 and 5 are not flushed or connected by means of the short-circuit line 13 when blood is detected by the blood leak sensor 11, the blood treatment apparatus 1000 comprises a monitoring device 31. The monitoring device 31 of FIG. 1 is designed as or comprises a connection sensor which detects a successfully established connection of lines 1 and 5 with the short-circuit line 13 and reports accordingly to a control circuit 33 by means of a connection signal. As the control circuit 33 in the embodiment shown in FIG. 1 is also connected with the blood leak sensor 11, which is indicated in the figures by means of broken lines, the information that there has been a blood leak, and the information that subsequently lines 1, 5 have been connected with the short-circuit line 13, concur (or are both received). Consequently, the control circuit 33 can, in interaction with the required devices, for example issue a warning to the user, start a cleaning procedure of the third and/or fourth connector 15, 17, block the blood treatment apparatus 1000 for patient treatment until further notice, or take other measures or emit warning hints.

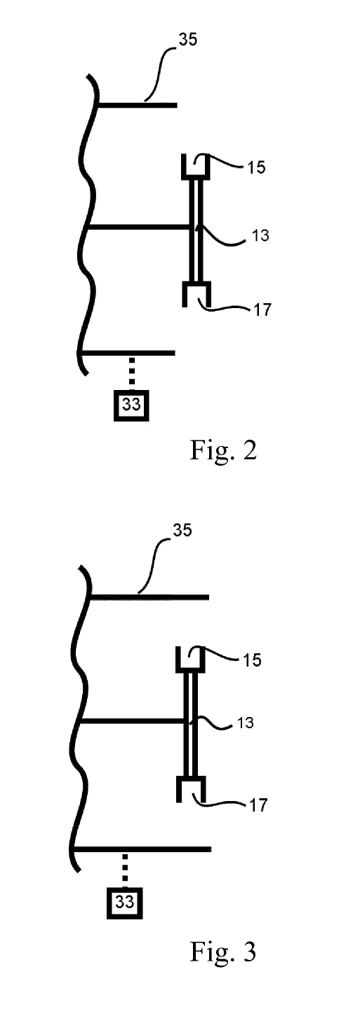

FIG. 2 shows in a schematically very simplified way sections of a blood treatment apparatus according to the present invention in a second embodiment. An interlock 35 can be seen, which is arranged to mechanically prevent a connection of connectors with the third and/or fourth connector 15, 17 of the short-circuit line 13, when the interlock 35 is in its cover or interlock state, which is shown in FIG. 3. In FIG. 2, however, the interlock 35 is shown in its connection state, in which such connection is not prevented.

FIG. 3 shows the subject-matter of FIG. 2. It can be seen that the interlock 35, which is shown in its interlock state, mechanically prevents connecting of connectors with the third and/or the fourth connector 15, 17 of the short-circuit line 13, as desired. In FIG. 3, this is achieved by relocating or blocking the access to the connectors 15, 17. Any other mechanical prevention of a connecting or a connection is also encompassed by the present invention.

Also the use of a flap (not shown here) which is foldable as a cover across the connectors 15, 17 and thus into an interlock state belongs to such mechanical prevention.

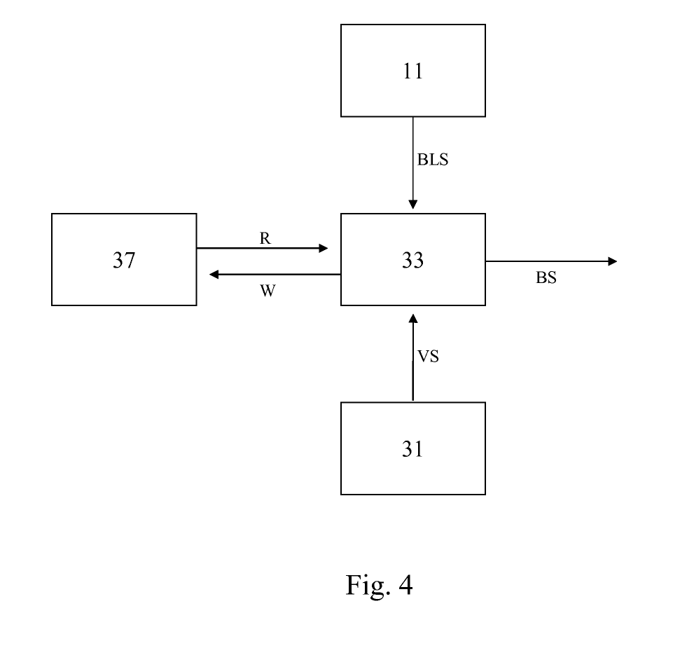

FIG. 4 shows the method according to the present invention in an exemplary embodiment.

In a first step, the blood leak sensor 11 detects the transition of blood from the blood side of the dialysis filter 100 into the dialysate side thereof, which triggers the output of a blood leak signal BLS to the control circuit 33.

If desired, the control circuit 33 or a storage device 37 which is connected herewith may buffer the blood leak signal BLS for a predetermined time period until the blood treatment session has been finished, for example by setting a so-called tag. If a storage device 37 is provided, the control circuit 33 is in reading and writing connection with it, by means of signals R and W.

If the monitoring device 31 detects the connection of the third and/or fourth connector 15, 17 with further connectors, for example the first or second connector 3, 7, in a later step, it emits a connection signal VS to the control device 33, which documents a successfully established connection.

The control device 33 continuously or in predefined intervals checks whether it has received and, if necessary, buffered both a blood leak signal BLS and a connection signal VS. If the result of this check is positive, i.e. both signals are present, the risk of a contamination of the third and/or fourth connector 15, 17 is to be assumed. In this case, the control device 33 initiates by means of an actuation signal BS one or several of the steps described herein for preventing risk. Among these are emitting a warning, starting a cleaning procedure, blocking the use of the blood treatment apparatus 1000 until further notice, or mechanically interlocking or covering the third and/or fourth connector 15, 17. The control device 33 is connected with the required apparatuses of the blood treatment apparatus 1000 or is in signal connection with them for this purpose.

### REFERENCE NUMERALS

| | |
|---|---|
| 1000 | blood treatment apparatus |
| 100 | dialysis filter |
| 1 | dialysis liquid inlet line |
| 3 | first connector |
| 5 | dialysate outlet line |
| 7 | second connector |
| 9 | intake |
| 11 | blood leak sensor |
| 13 | short-circuit line or short circuit |
| 15 | third connector |
| 17 | fourth connector |
| 119, 121 | connector of the dialysis filter on the dialysate side |
| 123, 125 | connector of the dialysis filter on the blood side |
| 31 | monitoring device |
| 33 | control or regulation (circuit) |
| 35 | interlock or cover |
| 37 | storage device |
| BLS | blood leak signal |
| BS | actuation signal |
| R | read signal |
| VS | connection signal |
| W | write signal |

1-12. (canceled)

13. A blood treatment apparatus comprising:
a dialysis filter or an intake for a dialysis filter;
a dialysis liquid inlet line with a first connector to feed dialysis liquid into the dialysis filter;
a dialysate outlet line with a second connector to discharge dialysate from the dialysis filter;
a blood leak sensor configured and arranged to at least one of detect blood in or at the dialysis filter, detect blood in the dialysis liquid inlet line, or detect blood in the dialysate outlet line, and configured to emit a blood leak signal if blood is detected;
a short-circuit line or a short-circuit unit to connect the dialysis liquid inlet line and, or with, the dialysate outlet line in fluid communication, wherein the short-circuit line comprises a third connector and a fourth connector, to be able to connect to one of the first and the second connectors, respectively;
a control circuit configured to receive the blood leak signal, and to initiate at least one step from the group consisting of:
emitting a warning to the user that cleaning at least one of the third or the fourth connector is required;
starting a prescribed or predetermined cleaning procedure of at least one of the third or the fourth connector;
stopping or prohibiting the operability of the blood treatment apparatus until the user has completed at least one prescribed activity; and
mechanically preventing a connection of at least one of the third or the fourth connector with at least one of the first or the second connector;
if the blood leak signal was at least one of emitted or received.

14. The blood treatment apparatus according to claim 13, further comprising a monitoring device configured to detect a connecting of at least one of the third or the fourth connector with at least one of the first or the second connector, and which is further configured to at least one of emit or receive a connection signal if connecting is detected, wherein the control circuit is configured to initiate at least one step from the group if or when both the blood leak signal and the connection signal have been at least one of emitted or received.

15. The blood treatment apparatus according to claim 14, wherein the monitoring device comprises a connection sensor configured and arranged to detect a successfully established connection with at least one of the third or the fourth connector, and wherein the monitoring device is configured to emit the connection signal if connecting is detected.

16. The blood treatment apparatus according to claim 13, wherein the blood treatment apparatus comprises or is a query device by means of which the user can or must at least one of confirm or deny a successfully established connection upon a query if the blood leak signal has been at least one of emitted or received.

17. The blood treatment apparatus according to claim 16, wherein in case of a confirmation, the treatment of the patient by means of the blood treatment apparatus cannot be continued or resumed or a warning is emitted to keep the treatment suspended or not to initiate the treatment again.

18. The blood treatment apparatus according to claim 13, wherein the at least one prescribed activity is chosen from a group consisting of inputting and performing cleaning steps by the user.

19. The blood treatment apparatus according to claim 13, further comprising an interlock or a cover which in an interlock or a cover state hereof covers at least one of the third or the fourth connector in a cover state such that connecting of the at least one interlocked or covered connector with the first or the second connector is not possible, and which is transferable in a connection state hereof, in which connecting is possible, wherein a mechanical prevention of connecting of at least one of the third or fourth connector with at least one of the first or second connector is a transfer of the interlock or the cover into the interlock or cover state, or retaining it herein.

20. The blood treatment apparatus according to claim 19, wherein the cover is a flap.

21. The blood treatment apparatus according to claim 13, further comprising a storage device which is configured or controlled to buffer the blood leak signal for a predetermined period or until a predetermined event takes places or starts.

22. The blood treatment apparatus according to claim 21, wherein the predetermined event is a subsequent patient's treatment.

23. A method to operate a blood treatment apparatus, the method comprising:
providing the blood treatment apparatus, the blood treatment apparatus comprising:
a dialysis filter or an intake for a dialysis filter;
a dialysis liquid inlet line with a first connector to feed dialysis liquid into the dialysis filter;
a dialysate outlet line with a second connector to discharge dialysate from the dialysis filter;
a blood leak sensor to at least one of detect blood in or at the dialysis filter, detect blood in the dialysis liquid inlet line, or detect blood in the dialysate outlet line, and to emit a blood leak signal if blood is detected;
a short-circuit line to connect the dialysis liquid inlet line and, or with, the dialysate outlet line in fluid communication, wherein the short-circuit line comprises a third connector and a fourth connector, to connect to one of the first and the second connectors, respectively; and
a control circuit;
detecting by means of the blood leak sensor whether a blood leak is present, and emitting a blood leak signal if the blood leak is present;
checking whether the blood leak signal has been at least one of emitted, received or processed;
if the blood leak signal has been at least one of emitted, received or processed, then initiating at least one step from the following group of steps:
emitting a warning to the user that cleaning at least one of the third or the fourth connector is necessary;
starting a predetermined cleaning procedure of at least one of the third or the fourth connector;
stopping or prohibiting the operability of the blood treatment apparatus until the user has completed at least one prescribed activity; and
mechanically preventing a connection of at least one of the third or the fourth connector with at least one of the first or the second connector.

24. The method according to claim 23, further comprising buffering the blood leak signal in a storage device.

25. The method according to claim 23, wherein the blood treatment apparatus further comprises a monitoring device to detect a connecting of at least one of the third or the fourth connector with at least one of the first or the second connector, and to emit a connection signal, wherein the method further comprises:
detecting by means of the monitoring device whether after output of the blood leak signal at least one of the third or the fourth connector has been connected, and emitting the connection signal if at least one of the third or the fourth connector has been connected;
wherein the control circuit is configured to initiate at least one step from the group if both the blood leak signal and the connection signal have been at least one of emitted, received or processed.

26. A method for replacing a dialysis filter of a blood treatment apparatus, the method comprising:
providing the blood treatment apparatus, the blood treatment apparatus comprising:
a first dialysis filter in an intake for a dialysis filter;
a dialysis liquid inlet line with a first connector to feed dialysis liquid into the first dialysis filter;
a dialysate outlet line with a second connector to discharge dialysate from the first dialysis filter;
a blood leak sensor to at least one of detect blood in or at the first dialysis filter, detect blood in the dialysis liquid inlet line, or detect blood in the dialysate outlet line, and to emit a blood leak signal if blood is detected; and
a short-circuit line to connect the dialysis liquid inlet line and, or with, the dialysate outlet line in fluid communication, wherein the short-circuit line comprises a third connector and a fourth connector, to connect to one of the first and the second connectors, respectively;
providing a second dialysis filter;
separating the dialysis liquid inlet line and the dialysate outlet line from the first dialysis filter; and
connecting the dialysis liquid inlet line and the dialysate outlet line with the second dialysis filter, without before having connected the dialysis liquid inlet line or the dialysate outlet line with the short-circuit line.
